(19) United States
(12) Patent Application Publication
(10) Pub. No.: US 2022/0273534 A1
(43) Pub. Date: Sep. 1, 2022

(54) LIQUID ORAL CARE COMPOSITION

(71) Applicant: Meda Pharma S.P.A., Monza (IT)

(72) Inventors: Andrea Zanardi, Milano (IT); Cleo Fabbro Guaetta, Livorno Li (IT); Elena Gelfi, Bergamo (IT); Marco Demurtas, Presezzo, Bergamo (IT)

(21) Appl. No.: 17/631,637

(22) PCT Filed: Jul. 15, 2020
(86) PCT No.: PCT/EP20/70039

§ 371 Date: Jan. 31, 2022

Related U.S. Application Data
(60) Provisional application No. 62/881,559, filed on Aug. 1, 2019.

(51) Int. Cl. A61K 8/20; A61K 8/27; A61K 8/41; A61K 8/43; A61Q 11/00

(52) U.S. Cl. CPC A61K 8/20; A61K 8/27; A61K 8/41; A61K 8/43; A61Q 11/00

(57) ABSTRACT

Described is a liquid oral care composition that includes an antihalitosis agent including chlorhexidine or a salt thereof and a buffering agent including a pH buffer having at least three hydroxyl groups, such as tromethamine. Also described is the use of a pH buffer having at least three hydroxyl groups, such as tromethamine, for stabilizing chlorhexidine or a salt thereof in a liquid oral care composition

### CROSS-REFERENCE TO RELATED APPLICATIONS

This application is the United States national phase of International Application No. PCT/EP2020/070039 filed Jul. 15, 2020, and claims the priority benefit under 35 U.S.C. 119(e) of U.S. Provisional Application No. 62/881,559 filed Aug. 1, 2019, the entire disclosures of which are hereby incorporated by reference herein in their entirety.

### FIELD OF THE INVENTION

The present disclosure relates to a liquid oral care composition comprising a pH buffer, such as tromethamine, which is effective in improving the stability of one or more active ingredients of the liquid oral care composition.

### BACKGROUND

Oral hygiene is an important consideration in the daily lives of children and adults around the globe. The maintenance of good oral hygiene is vital to keep the mouth, teeth, and gums free from disease, and to prevent the growth of bacteria which can lead to the development of halitosis (bad breath).

Bacteria located in periodontal pickets and at the back of the tongue break down sulfur-containing amino acids, in particular cysteine and methionine, to produce volatile sulfur compounds, including hydrogen sulfide and methyl mercaptan.

These volatile sulfur compounds have an extremely unpleasant odor, even at low concentrations, and also penetrate the epithelium and cause damage to cells of the underlying tissue. It has also been suggested that volatile sulfur compounds produced by bacteria located within periodontal pockets may be an important factor in the development of periodontal disease.

Antibacterial agents are often used in preventive dentistry to reduce the levels of these bacteria and thereby prevent the formation of volatile sulfur compounds. One known antibacterial agent used in preventative dentistry is chlorhexidine, which is frequently formulated in a mouthwash.

WO 00/051559 discloses a mouthwash comprising chlorhexidine and zinc acetate, wherein the combination of zinc and chlorhexidine synergistically reduces the production of volatile sulfur compounds.

However, chlorhexidine can be unstable in mouthwash formulations, specifically at neutral pH values, and degrades overtime. This leads to a decrease in pH of the mouthwash formulation and precipitation of the active ingredients. Such degradation is clearly undesirable because the efficacy of the product will be reduced and form undesired degradation products.

### SUMMARY OF THE INVENTION

In a first aspect, the invention provides a liquid oral care composition, comprising: an antihalitosis agent comprising chlorhexidine or a salt thereof; and a buffering agent comprising a pH buffer having at least three hydroxyl groups.

In a second aspect, the invention provides a liquid oral care composition comprising:
chlorhexidine or a salt thereof; and
tromethamine or a derivative of tromethamine; and optionally
a metal salt.

In a third aspect, the invention provides a liquid oral care composition as defined by the first or second aspect of the invention for use in therapy.

As will become apparent from this disclosure, the use of a pH buffer having at least three hydroxyl groups has particular application in stabilizing chlorhexidine or a salt thereof in a liquid oral care composition.

Accordingly, the invention is also directed to the use of a pH buffer having at least three hydroxyl groups for stabilizing chlorhexidine or salt thereof in a liquid oral care composition.

Additional aspects of the invention are more fully described in the following detailed description of the various embodiments.

### Definitions

For purposes of the following detailed description, it is to be understood that the invention may assume various alternative variations and step sequences, except where expressly specified to the contrary. Moreover, other than in any operating examples, or where otherwise indicated, all numbers expressing, for example, quantities of ingredients used in the specification and claims are to be understood as being modified in all instances by the term “about”. Accordingly, unless indicated to the contrary, the numerical parameters set forth in the following specification and attached claims are approximations that may vary depending upon the desired properties to be obtained by the present invention. At the very least, and not as an attempt to limit the application of the doctrine of equivalents to the scope of the claims, each numerical parameter should at least be construed in light of the number of reported significant digits and by applying ordinary rounding techniques.

All documents, such as but not limited to issued patents and patent applications, referred to herein, and unless otherwise indicated, are to be considered to be “incorporated by reference” in their entirety.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard variation found in their respective testing measurements.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

In this application, the use of the singular includes the plural and plural encompasses singular, unless specifically stated otherwise. In addition, in this application, the use of “or” means “and/or” unless specifically stated otherwise, even though “and/or” may be explicitly used in certain instances. Further, in this application, the use of “a” or “an” means “at least one” unless specifically stated otherwise. For example, “a” salt, “an” anticaries agent, “a” pH buffer, and the like refer to one or more of any of these items.

The proportions of each component of the liquid composition used herein are defined as “% w/w”, i.e. weight by weight, which is calculated by the following formula: % w/w=[(weight of solute)/(total weight of solution)]*100. If the relevant solute is added in the form of a salt, the weight of the solute is the weight of the salt before it is added to the liquid composition i.e. the anion(s) and the cation(s).

### DETAILED DESCRIPTION OF THE INVENTION

### Compositions

The present disclosure is directed to the use of a pH buffer having at least three hydroxyl groups for stabilizing chlorhexidine or salt thereof in a liquid oral care composition. The disclosure is based on the unexpected finding that a pH buffer having at least three hydroxyl groups can be used to stabilize chlorhexidine in a liquid oral care composition. In one non-limiting embodiment, the pH buffer having at least three hydroxyl groups is tromethamine. Accordingly, the disclosure is also directed to the use of tromethamine for stabilizing chlorhexidine or a salt thereof in a liquid oral care composition.

The invention provides a liquid oral care composition, comprising: an antihalitosis agent comprising chlorhexidine or a salt thereof; and a buffering agent comprising a pH buffer having at least three hydroxyl groups.

### Chlorhexidine

Chlorhexidine is a bis-biguanide with the chemical name N,N″-bis(4-chlorophenyI)-3,12-diimino-2,4,11,13-etraazatetradecanediimidamide), as depicted in Formula I:

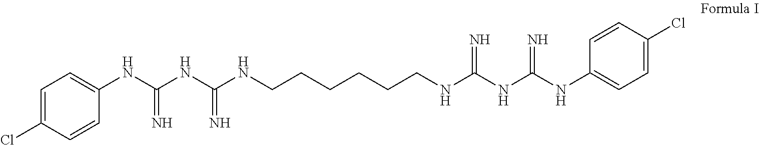 [SMILES: N=C(NCCCCCCNC(=N)NC(=N)Nc1ccc(Cl)cc1)NC(=N)Nc1ccc(Cl)cc1]

Chlorhexidine is an antibacterial agent used in oral care applications.

In some non-limiting embodiments, the liquid oral care composition includes a salt of chlorhexidine. Non-limiting examples of suitable salts of chlorhexidine that can be used in the liquid oral care compositions of the present disclosure include chlorhexidine diacetate, chlorhexidine dihydrochloride, chlorhexidine digluconate, or mixtures thereof.

Chlorhexidine or a salt thereof can comprise at least about 0.005% w/w, at least about 0.01% w/w, or at least about 0.02% w/w of the liquid oral care composition. Chlorhexidine or a salt thereof can comprise up to about 1% w/w, up to about 0.5% w/w, up to about 0.3% w/w, up to about 0.1% w/w, up to about 0.08% w/w, up to about 0.05% w/w, or up to about 0.04% w/w of the liquid oral care composition.

For example, the liquid oral care compositions of the present disclosure can comprise chlorhexidine or a salt thereof within a range such as from about 0.005% to about 1% w/w, about 0.005% to about 0.5% w/w, about 0.01% to about 0.3% w/w, about 0.01% to about 0.08% w/w, about 0.01% to about 0.05% w/w, about 0.02% to about 0.04% w/w, or about 0.005% to about 0.1% w/w of the liquid oral care composition.

In particular, the composition includes chlorhexidine diacetate. In one embodiment, the composition comprises 0.005 to 1% w/w, 0.005 to 0.5% w/w, 0.005 to 0.1% w/w, 0.01 to 0.3% w/w, 0.01 to 0.08% w/w, 0.01 to 0.05% w/w, or 0.02 to 0.04% w/w chlorhexidine diacetate. In particular, the composition includes 0.02 to 0.04% w/w chlorhexidine diacetate e.g 0.03% w/w chlorhexidine diacetate.

Chlorhexidine has a prolonged and broad spectrum antimicrobial effect, as well as plaque inhibitory potential. See Vrani “Formulation ingredients for toothpastes and mouthwashes” Bosnian Journal of Basic Medical Sciences 2004, 4, 51-58. The antimicrobial effect reduces offensive oral odor caused by volatile sulfur compounds. See N. Lourith “Oral malodour and active ingredients for treatment” International Journal of Cosmetic Science 2010, 32, 321-329.

Chlorhexidine is an amphipathic molecule with hydrophilic and hydrophobic groups. At physiological pH, chlorhexidine is cationic and exhibits its antimicrobial activity as a “membrane-active agent” that binds to negatively charged bacterial cell walls. Chlorhexidine establishes a bridge between pairs of adjacent phospholipids within the cell wall and displaces the associated divalent cations, thereby increasing the permeability of the cell membrane, facilitating the release of intracytoplasmic material and ultimately resulting in cell death. The cationic molecule will bind principally to anionic compounds within the cell wall, such as free sulphates, lipopolysaccharide phosphate groups and protein carboxyl groups. See Rölla and Melsen “On the mechanism of the plaque inhibition by chlorhexidine” Journal of Dental Research 1975, 54, 57-62.

However, chlorhexidine and chlorhexidine salts can suffer from hydrolytic degradation. It is known that in acidic conditions chlorhexidine degrades by two major pathways: one that leads to direct formation of its primary degradation product p-chloroaniline (PCA) and the other that leads to indirect formation of p-chloroaniline via the formation of the intermediate (p-chlorophenyl)biguanide-N-amidino-N′-(p-chlorophenyl)urea (PBG-PAU) with loss of ammonia. In alkaline conditions, chlorhexidine degrades by a different single pathway leading indirectly to p-chloroaniline. Postulated chlorhexidine degradation mechanisms are set out in Zong “Studies on the Instability of Chlorhexidine, Part I: Kinetics and Mechanisms” Journal of Pharmaceutical Sciences 2012, 101, 2417-2427. This decomposition pathway can occur in aqueous oral care compositions.

### The Buffering Agent

The liquid oral care composition of the present disclosure includes a buffering agent that includes a pH buffer having at least three hydroxyl groups, such as tromethamine. Such a buffering agent has been found to improve the chemical stability of chlorhexidine or a salt thereof in a liquid oral care composition.

Non-limiting examples of suitable pH buffers having at least three hydroxyl groups include tris(hydroxymethyl)aminomethane (tromethamine), [tris(hydroxymethyl)methylamino]propanesulfonic acid (TAPS), 3-[N-Tris(hydroxymethyl)methylamino]-2-hydroxypropanesulfonic acid (Tricine), 3-[N-Tris(hydroxymethyl)methylamino]-2-hydroxypropanesulfonic acid (TAPSO), and 2-[[1,3-dihydroxy-2-(hydroxymethyl)propan-2-yl]amino]ethanesulfonic acid (TES). For example, TAPS is generally useful in a pH range of about 7.7 to about 9.1, Tricine is generally useful in a pH range of about 7.4 to about 8.8, TABSO is generally useful in a pH range of about 7.0 to about 8.2, and TES is generally useful in a pH range of about 6.8 to about 8.2.

In one particular non-limiting embodiment, the buffering agent includes tromethamine or a derivative of tromethamine. Tromethamine includes a primary amine group and three hydroxyl groups and is depicted in Formula II:

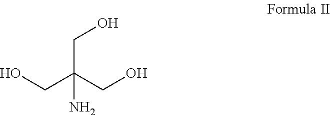

It should be noted that the term tromethamine includes salts of tromethamine, and the compositions can be prepared by including tromethamine salts, such as tromethamine hydrochloride. In one embodiment, the buffering agent is a salt of tromethamine.

Tromethamine can comprise at least about 0.1% w/w, at least about 0.5% w/w, or at least about 1% w/w of the liquid oral care composition. Tromethamine can comprise up to about 3% w/w, up to about 2.5% w/w, up to about 2% w/w, up to about 1.5% w/w, up to about 1.25% w/w, or up to about 1.21% w/w of the liquid oral care composition. For example, the liquid oral care compositions of the present disclosure can include tromethamine within a range such as from about 0.1% to about 3% w/w, from about 0.5% to about 2.5% w/w, from about 0.5% to about 1.5% w/w, from about 0.5% to about 1.25% w/w, or from about 0.5% to about 1.21% w/w of the liquid oral care composition.

It has been found that the presence of the above-described pH buffer unexpectedly provides improved chemical stability of chlorhexidine or a salt thereof within the oral care composition. In other words, the amount of chlorhexidine that chemically decomposes is less in the presence of the pH buffer. As such, the chlorhexidine or a salt thereof remains in solution rather than precipitating over time, and the liquid oral care composition is able to maintain a constant pH for prolonged periods.

By reducing the rate of degradation of chlorhexidine, the antibacterial effect of the active ingredient is prolonged and the shelf life of the liquid oral care composition is extended.

The buffering agent may or may not include additional pH buffers. For example, the buffering agent may additionally include citric acid and/or benzoic acid. In certain non-limiting embodiments, the buffering agent includes greater than about 50 weight % tromethamine, greater than about 75 weight % tromethamine, or greater than about 95 weight % tromethamine (including 100 weight % tromethamine) based on the total weight of the buffering agent.

The buffering agent can comprise at least about 0.1% w/w, at least about 0.5% w/w, or at least about 1% w/w of the liquid oral care composition. The buffering agent can comprise up to about 7% w/w, up to about 5% w/w, or up to about 3% w/w of the liquid oral care composition. For example, the liquid oral care composition can include the buffering agent within a range such as from about 0.1% to about 7% w/w, from about 0.1% to about 5% w/w, from about 0.5% to about 5% w/w, or from about 1% to about 3% w/w of the liquid oral care composition.

The buffering agent may be present in an amount such that the pH of the oral care preparation, at the time of preparation, is in the range of from about 6 to about 8, from about 6 to about 7.5, from about 6.5 to about 7.5, from about 6.5 to about 7.25, or from about 6.5 to about 7.

### The Metal Salt

The present disclosure is also directed to a liquid oral care composition that includes an antihalitosis agent comprising chlorhexidine or a salt thereof and a buffering agent comprising a pH buffer having at least three hydroxyl groups.

As mentioned above, the liquid oral care composition of the present disclosure can include an antihalitosis agent that may include chlorhexidine or a salt thereof. An “antihalitosis agent,” as used herein, refers to a compound or substance which acts as an antimicrobial agent to reduce malodor.

The antihalitosis agent can comprise at least about 0.005% w/w, at least about 0.01% w/w, or at least about 0.1% w/w of the liquid oral care composition. The antihalitosis agent can comprise up to about 1% w/w, up to about 0.9% w/w, or up to about 0.8% w/w of the liquid oral care composition. For example, the liquid oral care composition can comprise the antihalitosis agent within a range such as from about 0.005% w/w to about 1% w/w, from about 0.01% w/w to about 0.9% w/w, or from about 0.1% w/w to about 0.8% w/w.

The antihalitosis agent may further comprise a metal salt. The metal salt may be the salt of a divalent metal such as, for example, a zinc salt, a tin salt, a copper salt, a potassium salt, or mixtures thereof.

In one non-limiting embodiment, the metal salt is a zinc salt. Non-limiting examples of suitable zinc salts include zinc oxide, zinc lactate, zinc chloride, zinc citrate, zinc acetate, zinc borate, zinc butyrate, zinc carbonate, zinc formate, zinc gluconate, zinc glycerate, zinc glycolate, zinc phosphate, zinc picolinate, zinc proprionate, zinc salicylate, zinc silicate, zinc stearate, zinc tartrate, zinc undecylenate, zinc phosphate, zinc ricinoleate, zinc nitrate or zinc sulfate or mixtures thereof. In one embodiment, the zinc salt is zinc acetate. In one non-limiting embodiment, the zinc acetate is zinc acetate dihydrate.

Zinc ions reduce the production of volatile sulfur compounds in the oral cavity. It is thought that a reaction between zinc and sulfur produces a non-volatile sulfide product, thereby preventing the transformation of sulfur containing substrates to volatile sulfur compounds. In addition, zinc ions possess certain antibacterial activity known to inhibit plaque formation and reduce acid formation in dental plaque. See “Oral malodour and active ingredients for treatment”, International Journal of Cosmetic Science, 2010, 32, 321-329. The combination of zinc acetate and chlorhexidine provides a synergistic effect against the production of volatile sulfur compounds, as described in U.S. Pat. No. 6,344,184, which disclosure is incorporated herein by reference.

The metal salt can comprise at least about 0.1% w/w, at least about 0.2% w/w, at least about 0.3% w/w, or at least about 0.4% w/w of the liquid oral care composition. The metal salt can comprise up to about 1% w/w, up to about 0.9% w/w, up to about 0.8% w/w, up to about 0.7% w/w, or up to about 0.5% w/w of the liquid oral care composition.

For example, the liquid oral care compositions of the present disclosure can include a metal salt within a range such as from about 0.1% to about 1% w/w, about 0.2% to about 0.9% w/w, about 0.3% to about 0.8% w/w, about 0.4% to about 0.7% w/w, or about 0.2% to about 0.5% w/w, such as about 0.3% w/w of the liquid oral care composition.

In one embodiment, the liquid oral care composition comprises 0.1 to 1% w/w zinc salt. Alternatively, the composition contains 0.2 to 0.9% w/w, 0.3 to 0.8% w/w or 0.4 to 0.7% w/w zinc salt. In a further embodiment, the composition contains 0.2 to 0.5% w/w zinc salt. In a preferred embodiment, the composition contains 0.3% w/w zinc salt.

In one embodiment, the liquid oral care composition comprises 0.1% to 1% w/w zinc acetate. Alternatively, the composition contains 0.2 to 0.9% w/w, 0.3 to 0.8% w/w or 0.4 to 0.7% w/w zinc acetate. In a further embodiment, the composition contains 0.2 to 0.5% w/w zinc acetate. In a preferred embodiment, the composition contains 0.3% w/w zinc acetate.

### The Compositions

In one non-limiting embodiment, the liquid oral care composition is in the form of a mouthwash, mouthrinse or a spray. In particular, the liquid oral care composition can be a mouthwash, for example an aqueous mouthwash.

### Other Components

The oral care composition that is a mouthwash, mouthrinse or a spray generally includes water. In addition, the oral care composition includes other additives and adjuvants commonly found in oral care compositions, for example an antiplaque agent, a masking agent, a humectant, a flavoring agent, and a buffering agent.

### Water

Typically, water (e.g., demineralized water) is present as the balance of the composition. In other words, the composition is an aqueous oral care composition. Such aqueous oral care compositions can, for example, include at least about 50% water, at least about 75% water, at least about 85% water, or at least about 95% water, based on the total weight of the liquid medium. Additionally, other solvents may be present in the liquid oral care composition, such as alcohol solvents. For example, the liquid oral care composition may optionally include ethanol.

### Anticaries Agent

The liquid oral care composition of the present invention may also include an anticaries agent. An “anticaries agent,” as used herein, is a compound or substance which prevents the formation of caries by inhibiting tooth decay.

The anticaries agent may include one or more fluoride salts. Non-limiting examples of fluoride salts include stannous fluoride, sodium fluoride, potassium fluoride, potassium monofluorophosphate, sodium monoflourophosphate, ammonium monoflourophosphate, sodium flourosilicate, ammonium flourosilicate, amine fluorides such ammonium fluoride, and combinations thereof.

In one non-limiting embodiment, the oral care composition includes an amount of a fluoride salt such that the composition includes fluoride ions in an amount of from about 50 to about 5000 ppm, such as from about 100 to about 1000 ppm or from about 200 to about 500 ppm. In one particular example, the oral care composition includes an amount of a fluoride salt such that the composition includes about 250 ppm fluoride ions.

The anticaries agent can comprise at least about 0.005% w/w, at least about 0.01% w/w, or at least about 0.2% w/w of the liquid oral care composition. The anticaries agent can comprise up to about 1% w/w, up to about 0.1% w/w, or up to about 0.07% w/w of the liquid oral care composition. For example, the liquid oral care compositions can include the anticaries agent within a range such as from about 0.01% to about 1% w/w, from about 0.01% to about 0.1% w/w, or from about 0.02% to about 0.07% w/w, such as about 0.05% w/w of the liquid oral care composition.

### Masking Agent

The liquid oral care composition of the present disclosure can also include a masking agent. As used herein, the term “masking agent” refers to an agent which reduces the aftertaste of any minerals present in the oral care composition. Non-limiting examples of suitable masking agents include tannic acid, glycyrrhizin derivatives, and acesulfame, such as potassium acesulfame. The liquid oral care compositions can comprise the masking agent in a range from about 0.01 to about 0.1% w/w or from about 0.01 to about 0.05% w/w.

### Humectant

The liquid oral care composition can also include a humectant. As used herein, the term “humectant” refers to a hygroscopic substance which retains moisture and increases the viscosity of the liquid oral care composition. The humectant provides a certain mouth feel to the oral care composition. Non-limiting examples of suitable humectants include hydrogenated starch hydrolysate, glycerin, sorbitol, and combinations thereof. The liquid oral care composition of the present disclosure can comprise the humectant in a range from about 1 to about 25% w/w or from about 1 to about 20% w/w.

### Flavouring Agent

The liquid oral care composition of the present disclosure can further include a flavoring agent. Non-limiting examples of suitable flavoring agents include peppermint, spearmint, cinnamon, wintergreen oils, menthol, methyl salicylate derivatives, eucalyptus, and combinations thereof. The liquid oral care composition can comprise the flavoring agent in a range from about 0.1 to about 1% w/w or from about 0.2 to about 0.7% w/w.

### Emulsifying Agent

The liquid oral care composition can also include an emulsifying agent. As used herein, the term “emulsifying agent” refers to a compound or substance that acts as a stabilizer for emulsions. Non-limiting examples of suitable emulsifying agents include hydrogenated castor oil, polysorbate-20, polyethylene glycol, and combinations thereof. The liquid oral care composition can include the emulsifying agent in a range from about 0.1 to about 10% w/w or from about 0.1 to about 5% w/w. Certain of these compounds can also be useful as a solubilizing/solvent agent. For example, PEG-40 can be used to solubilize an aroma in the composition.

### Other Components

The liquid oral care composition may include various other additives or adjuvants to make the liquid oral care composition more amenable for use in a particular application without negatively affecting the efficacy or the stability of chlorhexidine in a substantial matter. Non-limiting examples of additives that can be used with the liquid oral care composition include emollients, fragrances, pigments, dyes, flavors, abrasives, bleaching agents, preservatives, antioxidants, and the like.

### Other Compositions

### Further Aspects of the Invention

In certain embodiments, liquid oral care compositions of the present disclosure can be prepared having the following composition:

TABLE 1

| Component | (% w/w) |
|---|---|
| Water | Up to 100 |
| Chlorhexidine Diacetate | 0.01-1% |
| Zinc Acetate | 0.1-1% |
| Potassium Acesulfame | 0.01-0.1% |
| Sodium Fluoride | 0.005-1% |
| Hydrogenated Starch Hydrolysate | 5-10% |
| Ethanol | 0-2% |
| Flavoring | 0.1-1% |
| PEG-40 Hydrogenated Castor Oil | 0.1-10% |
| Glycerin | 7-13% |
| Citric Acid | q.s. to pH 6.5-7.5 |
| Tromethamine | 0.1-2.5% |

Other compositions that form part of the invention include the following:

In a fourth aspect, the invention provides a liquid oral care composition (e.g. an aqueous oral care composition, in particular a mouthwash or spray) comprising:
chlorhexidine or a salt thereof;
tromethamine or a derivative of tromethamine; and
zinc acetate.

In a fifth aspect, the invention provides a liquid oral care composition comprising:
chlorhexidine or a salt thereof;
tromethamine or a derivative of tromethamine; and
a zinc salt,
   wherein the liquid oral care composition is an aqueous solution, and the aqueous solution has a pH of from 6 to 7.5.

In a sixth aspect, the invention provides a liquid oral care composition (e.g. a mouthwash or a spray) comprising:
chlorhexidine or a salt thereof;
tromethamine or a derivative of tromethamine; and
a zinc acetate,
   wherein the liquid oral care composition is an aqueous solution, and the aqueous solution has a pH of from 6 to 7.5.

In a seventh aspect, the invention provides a liquid oral care composition (e.g. an aqueous oral care composition, for example a mouthwash or spray) comprising:
chlorhexidine or a salt thereof;
0.1% to 3% w/w tromethamine or a derivative of tromethamine; and
a zinc salt.

In a eighth aspect, the invention provides a liquid oral care composition comprising:
chlorhexidine or a salt thereof;
tromethamine or a derivative of tromethamine; and
a zinc salt,
   wherein the composition is an aqueous mouthwash.

In a ninth aspect, the invention provides a liquid oral care composition comprising:
chlorhexidine or a salt thereof;
tromethamine or a derivative of tromethamine; and
a zinc salt,
   wherein the composition is an aqueous spray.

It should be understood that the above aspects are general statements of compositions and can be combined with the other embodiments of the invention described herein.

### Preferred Compositions

### Further Aspects of the Invention

Particular compositions according to the invention include the following.

A liquid oral care composition that is a mouthwash, mouthrinse or a spray comprising:
0.005% to 1% w/w chlorhexidine or a salt thereof;
0.1% to 3% w/w tromethamine or a derivative of tromethamine; and
0.1 to 1% w/w zinc salt.

A liquid oral care composition that is a mouthwash, mouthrinse or a spray comprising:
0.005% to 1% w/w chlorhexidine or a salt thereof;
0.1% to 3% w/w tromethamine or a derivative of tromethamine; and
0.1 to 1% w/w zinc salt.
   wherein the that is a mouthwash, mouthrinse or spray is an aqueous solution, and the aqueous solution has a pH of from 6 to 7.5.

A liquid oral care composition that is a mouthwash, mouthrinse or a spray comprising:
0.005% to 1% w/w chlorhexidine or a salt thereof;
0.1% to 3% w/w tromethamine or a derivative of tromethamine; and
0.1 to 1% w/w zinc acetate.

A liquid oral care composition that is a mouthwash, mouthrinse or a spray comprising:
0.005% to 1% w/w chlorhexidine or a salt thereof;
0.1% to 3% w/w tromethamine or a derivative of tromethamine; and
0.1 to 1% w/w zinc acetate,
   wherein the mouthwash, mouthrinse or a spray is an aqueous solution, and the aqueous solution has a pH of from 6 to 7.5.

Further particular compositions according to the invention include the following.

A liquid oral care composition that is a mouthwash, mouthrinse or a spray comprising:
0.01% to 0.05% w/w chlorhexidine or a salt thereof;
0.1% to 3% w/w tromethamine or a derivative of tromethamine; and
0.2 to 0.5% w/w zinc salt.

A liquid oral care composition that is a mouthwash, mouthrinse or a spray comprising:
0.01% to 0.05% w/w chlorhexidine or a salt thereof;
0.1% to 3% w/w tromethamine or a derivative of tromethamine; and
0.2 to 0.5% w/w zinc salt.
   wherein the mouthwash, mouthrinse or spray is an aqueous solution, and the aqueous solution has a pH of from 6.5 to 7.5.

A liquid oral care composition that is a mouthwash, mouthrinse or a spray comprising:
0.01% to 0.05% w/w chlorhexidine or a salt thereof;
0.1% to 3% w/w tromethamine or a derivative of tromethamine; and
0.2 to 0.5% w/w zinc acetate.

A liquid oral care composition that is a mouthwash, mouthrinse or a spray comprising:
0.01% to 0.05% w/w chlorhexidine or a salt thereof;
0.1% to 3% w/w tromethamine or a derivative of tromethamine; and
0.2 to 0.5% w/w zinc acetate,
   wherein the mouthwash, mouthrinse or a spray is an aqueous solution, and the aqueous solution has a pH of from 6.5 to 7.5.

### Uses of the Invention

### Stabilisation of Chlorhexidine

The present disclosure is directed to the use of a pH buffer having at least three hydroxyl groups for stabilizing chlorhexidine or salt thereof in a liquid oral care composition. The disclosure is based on the unexpected finding that a pH buffer having at least three hydroxyl groups can be used to stabilize chlorhexidine in a liquid oral care composition. In one non-limiting embodiment, the pH buffer having at least three hydroxyl groups is tromethamine. Accordingly, the disclosure is also directed to the use of tromethamine for stabilizing chlorhexidine or a salt thereof in a liquid oral care composition.

The liquid oral care composition of the present disclosure can remain stable with a constant or near constant pH over time, during which time the active ingredients remain in solution.

For example, liquid oral care compositions according to the present disclosure have been found to maintain a constant pH (within about 0.1 of the original pH value) when stored for at least six months at temperatures in the range of about 4° C. to about 50° C., including at those times and temperatures reported in the Examples below.

Additionally, liquid oral care compositions according to the present disclosure have been found maintain the concentration of chlorhexidine in solution, and avoid or limit the precipitation of chlorhexidine out of solution.

### Use of the Mouthwash

The liquid oral care composition of the present disclosure is particularly useful for therapy.

In particular, the liquid oral care composition may be administered to a patient orally.

In one embodiment, the liquid oral care composition can be used for the treatment or prophylaxis of a bacterial infection.

In one non-limiting embodiment, a method of treating a bacterial infection can include administering the liquid oral care composition described herein to a patient in need thereof.

Moreover, the composition described herein generally include agents which reduce the formation of volatile sulfur compounds. Chlorhexidine, for example, has an antibacterial effect against the microorganisms that breakdown sulfur containing amino acids. The compositions described herein may also include zinc salts, which are thought to react with sulfur containing compounds to give non-volatile sulfur compounds.

Accordingly, the invention provides the use of the liquid oral care compositions for neutralising halitosis.

The following examples are presented to demonstrate the general principles of the invention of this disclosure. The invention should not be considered as limited to the specific examples presented. All parts and percentages in the examples are % w/w unless otherwise indicated.

### Examples

Example compositions according to the present disclosure (Ex. 1-3) and comparative example compositions (Comp. Ex. 1-3) were prepared as follows:

TABLE 2

| Component | | | | Comp. | Comp. | Comp. |
|---|---|---|---|---|---|---|
| (% w/w) | Ex. 1 | Ex 2 | Ex. 3 | Ex. 1 | Ex. 2 | Ex. 3 |
| Water | 74.12 | 76.85 | 78.06 | 77.94 | 78.77 | 79.27 |
| Chlorhexidine | 0.03 | 0.03 | 0.03 | 0.03 | 0.03 | 0.03 |
| Diacetate | | | | | | |
| Zinc Acetate | 0.30 | 0.30 | 0.30 | 0.30 | 0.30 | 0.30 |
| Potassium Acesulfame | 0.03 | 0.03 | 0.03 | 0.03 | 0.03 | 0.03 |
| Sodium Fluoride | 0.05 | 0.05 | 0.05 | 0.05 | 0.05 | 0.05 |
| Hydrogenated Starch | 7.50 | 7.50 | 7.50 | 7.50 | 7.50 | 7.50 |
| Hydrolysate | | | | | | |
| Ethanol | 1.20 | 1.20 | 1.20 | 1.20 | 1.20 | 1.20 |
| Flavoring | 0.50 | 0.50 | 0.50 | 0.50 | 0.50 | 0.50 |
| PEG-40 Hydrogenated | 1.00 | 1.00 | 1.00 | 1.00 | 1.00 | 1.00 |
| Castor Oil | | | | | | |
| Glycerin | 10.10 | 10.10 | 10.10 | 10.10 | 10.10 | 10.10 |
| Citric Acid | 2.75 | 0.02 | 0.02 | — | 0.02 | 0.02 |
| 50% sol | | | | | | |
| Tromethamine | 2.42 | 2.42 | 1.21 | — | — | — |
| Citric Acid | — | — | — | 0.19 | — | — |
| monohydrate | | | | | | |
| Sodium Phosphate | — | — | — | 1.16 | — | — |
| Dibasic Anhydrous | | | | | | |
| Arginine | — | — | — | — | 0.05 | — |
| Glycine | — | — | — | — | — | 0.10 |

### Stability Studies

Formulations according to Table 2 were then subjected to stability studies. The formulations were stored in a glass bottles at the temperatures indicated in Tables 3-5. The pH of the formulations was measured over time using a Seven Easy Mettler Toledo pH meter.

TABLE 3
Formulations stored at 25° C.

| | | | | | Comp. | Comp. | Comp. |
|---|---|---|---|---|---|---|---|
| | | Ex. 1 | Ex. 2 | Ex. 3 | Ex. 1 | Ex. 2 | Ex. 3 |
| Time | | (pH) | (pH) | (pH) | (pH)** | (pH) | (pH) |
| 0 | | 6.85 | 6.85 | 6.63 | — | 6.62 | 6.67 |
| 14 | days | 6.85 | 6.85 | 6.62 | — | 6.62 | 6.6 |
| 1 | month | 6.84 | 6.84 | 6.62 | — | 6.57 | 6.68 |
| 2 | months | 6.86 | 6.86 | 6.64 | — | 6.62 | 6.72 |
| 3 | months | 6.92 | 6.92 | 6.63 | — | 6.6 | 6.75 |
| 4 | months | 6.87 | 6.87 | 6.64 | — | 6.54 | 6.65 |
| 5 | months | 6.88 | 6.88 | 6.63 | — | 6.53 | 6.67 |
| 6 | months | 6.86 | 6.86 | 6.60 | — | 6.51 | 6.52 |
| 12 | months | 6.68 | 6.86 | 6.67 | — | 6.37 | 6.25 |

**Comp. Ex. 1 did not conform to preset pH guidelines

TABLE 4
Formulations stored at 4° C.

| | | | | | Comp. | Comp. | Comp. |
|---|---|---|---|---|---|---|---|
| | | Ex. 1 | Ex. 2 | Ex. 3 | Ex. 1 | Ex. 2 | Ex. 3 |
| Time | | (pH) | (pH) | (pH) | (pH)** | (pH) | (pH) |
| 0 | | 6.85 | 6.85 | 6.63 | — | 6.62 | 6.67 |
| 14 | days | 6.84 | 6.84 | 6.62 | — | 6.63 | 6.68 |
| 1 | month | 6.84 | 6.84 | 6.63 | — | 6.62 | 6.7 |
| 2 | months | 6.81 | 6.81 | 6.67 | — | 6.67 | 6.74 |
| 3 | months | 6.92 | 6.92 | 6.68 | — | 6.7 | 6.78 |
| 4 | months | 6.86 | 6.86 | 6.64 | — | 6.61 | 6.7 |
| 5 | months | 6.88 | 6.88 | 6.64 | — | 6.66 | 6.73 |
| 6 | months | 6.86 | 6.86 | 6.61 | — | 6.63 | 6.69 |
| 12 | months | 6.89 | 6.89 | 6.66 | — | 6.55 | 6.52 |

**Comp. Ex. 1 did not conform to preset pH guidelines

TABLE 5
Formulations stored at 40° C.

| | | | | | Comp. | Comp. | Comp. |
|---|---|---|---|---|---|---|---|
| | | Ex. 1 | Ex. 2 | Ex. 3 | Ex. 1 | Ex. 2 | Ex. 3 |
| Time | | (pH) | (pH) | (pH) | (pH)** | (pH) | (pH) |
| 0 | | 6.85 | 6.85 | 6.63 | — | 6.62 | 6.67 |
| 14 | days | 6.83 | 6.83 | 6.61 | — | 6.56 | 6.64 |
| 1 | month | 6.83 | 6.83 | 6.61 | — | 6.54 | 6.63 |
| 2 | months | 6.83 | 6.83 | 6.63 | — | 6.55 | 6.62 |
| 3 | months | 6.92 | 6.92 | 6.69 | — | 6.55 | 6.64 |
| 4 | months | 6.85 | 6.85 | 6.65 | — | 6.44 | 6.53 |
| 5 | months | 6.87 | 6.87 | 6.65 | — | 6.46 | 6.53 |
| 6 | months | 6.85 | 6.85 | 6.62 | — | 6.4 | 6.47 |

**Comp. Ex. 1 did not conform to preset pH guidelines

As shown in Tables 3-5, the liquid oral care compositions of Examples 1-3, which contained tromethamine, maintain a pH within a range of 6.5 to 7.5 for at least 6 or 12 months when stored at room temperature (25° C.), low temperatures (4° C.), and elevated temperatures (40° C.). Further, the liquid oral care compositions of these examples maintained a pH value of ±0.1 of that of the starting pH value for at least 6 or 12 months when stored at 25° C., 4° C., or 40° C. On the other hand, Comparative Examples 1-3, which did not include tromethamine, either did not conform to the required pH level (Comp. Ex. 1) or exhibited greater pH variability over time than did Examples 1-3 (Comp. Exs. 2-3).

The stability of the formulations was also assessed through a chlorhexidine assay after storage at 50° C. for one month. The formulations were stored in glass bottles and the chlorhexidine assay was performed by HPLC-UV to measure the amount of chlorhexidine in the liquid oral care composition as compared to the initial level in the formulation.

TABLE 6
Chlorhexidine Assay

| | | | | Comp. | Comp. | Comp. |
|---|---|---|---|---|---|---|
| | Ex. 1 | Ex. 2 | Ex. 3 | Ex. 1 | Ex. 2 | Ex. 3 |
| Chlorhexidine | 90% | 90% | 90% | — | 87% | 40% |
| Remaining in | | | | | | |
| Solution | | | | | | |

** Comp. Ex. 1 did not conform to preset pH guidelines

As shown in Table 6, in the liquid oral care compositions of Examples 1-3, 90% of the original chlorhexidine remained in solution when stored at elevated thermal conditions (50° C.) for one month. In Comparative Examples 2-3, less chlorohexidine remained in solution, indicating that a greater amount of chlorohexidine precipitated out of solution in Comparative Examples 2-3.

Another example composition according to the present disclosure (Ex. 4) was prepared as follow:

TABLE 7

| | Ex. 4 |
|---|---|
| Component | (% w/w) |
| Water | 76.85 |
| Chlorhexidine Diacetate | 0.03 |
| Zinc Acetate | 0.3 |
| Potassium Acesulfame | 0.03 |
| Sodium Fluoride | 0.05 |
| Hydrogenated Starch Hydrolysate | 7.5 |
| Alcohol (ethanol) | 1.2 |
| Flavoring | 0.50 |
| PEG-40 Hydrogenated Castor Oil | 1.00 |
| Glycerin | 10.05 |
| Citric Acid (50% aq.) | to pH 7 |
| Tromethamine | 2.42 |

A first sample of the composition of Example 4 was packaged in a glass bottle and subjected to cosmetic stability tests under the following thermostat controlled conditions:

TABLE 8
Test conditions

| | | |
|---|---|---|
| Temperature | Relative Humidity | Duration |

| | | | | |
|---|---|---|---|---|
| 40° | C. | NA | 6 | months |
| 25° | C. | 60% | 12 | months |
| 4° | C. | NA | 6 | months |
| 50° | C. | NA | 1 | month |

The following stability data was gathered for this first sample of Example 4:

TABLE 9
Stability testing for 25° C. - 60% HR

| Test 25° C. - 60% HR | | Months | | | | | |
|---|---|---|---|---|---|---|---|
| Test | Specification | 0 | 1 | 2 | 3 | 6 | 12 |
| Aspect | Colorless liquid | Compliant | Compliant | Compliant | Compliant | Compliant | Compliant |
| Odor | typical | Compliant | Compliant | Compliant | Compliant | Compliant | Compliant |
| pH | 6.5-7.5 | 6.85 | 6.84 | 6.86 | 6.92 | 6.86 | 6.86 |

TABLE 10
Stability testing for 4° C.

| Test 4° C. | | Months | | | | |
|---|---|---|---|---|---|---|
| Test | Specification | 0 | 1 | 2 | 3 | 6 |
| Aspect | Colorless liquid | Compliant | Compliant | Compliant | Compliant | Compliant |
| Odor | typical | Compliant | Compliant | Compliant | Compliant | Compliant |
| pH | 6.5-7.5 | 6.85 | 6.84 | 6.81 | 6.92 | 6.86 |

TABLE 11
Stability testing for 40° C.

| Test 40° C. | | Months | | | | |
|---|---|---|---|---|---|---|
| Test | Specification | 0 | 1 | 2 | 3 | 6 |
| Aspect | Colorless liquid | Compliant | Compliant | Compliant | Compliant | Compliant |
| Odor | typical | Compliant | Compliant | Compliant | Compliant | Compliant |
| pH | 6.5-7.5 | 6.85 | 6.83 | 6.83 | 6.92 | 6.85 |

TABLE 12
Stability testing for 50° C.

| | |
|---|---|
| Test 50° C. | Months |

| Test | Specification | 0 | 1 |
|---|---|---|---|
| Aspect | Colorless liquid | Compliant | Compliant |
| Color | transparent | Compliant | Compliant |
| Odor | typical | Compliant | Compliant |
| pH | 6.5-7.5 | 6.85 | 6.85 |

A second sample of the composition of Example 4 was packaged in glass bottles and HDPE bottles and subject to a stability protocol provided by the International Council for Harmonisation of Technical Requirements for Pharmaceuticals for Human Use (ICH). The ICH stability protocol was performed under the following thermostat controlled conditions:

TABLE 13
Test conditions

| Temperature | Relative Humidity | Duration |
|---|---|---|
| 25° C. | 60% | 30 months |
| 30° C. | 65% | 12 months |
| 40° C. | 75% | 12 months |

The following stability data was gathered for this second sample of Example 4:

TABLE 14
Stability testing for 25° C. - 60% HR

| Test 25° C. - 60% HR | | Months | | | | | |
|---|---|---|---|---|---|---|---|
| Test | Specification | 0 | 1 | 2 | 3 | 6 | 12 |
| Aspect | Colorless liquid | Compliant | Compliant | Compliant | Compliant | Compliant | Compliant |
| Odor | typical | Compliant | Compliant | Compliant | Compliant | Compliant | Compliant |
| pH | 6.5-7.5 | 7.0 | 7.0 | 7.0 | 7.0 | 6.9 | 7.0 |
| Chlorhexidine | 0.24-0.36 | 0.31 | 0.31 | 0.30 | 0.32 | 0.31 | 0.31 |
| | mg/ml | | | | | | |
| Sodium fluoride | 0.45-0.55 | 0.50 | 0.50 | 0.50 | 0.51 | 0.52 | 0.51 |
| | mg/ml | | | | | | |

TABLE 15
Stability testing for 30° C. - 65% HR

| Test 30° C. - 65% HR | | Months | | | | | |
|---|---|---|---|---|---|---|---|
| Test | Specification | 0 | 1 | 2 | 3 | 6 | 12 |
| Aspect | Colorless liquid | Compliant | Compliant | Compliant | Compliant | Compliant | Compliant |
| Odor | typical | Compliant | Compliant | Compliant | Compliant | Compliant | Compliant |
| pH | 6.5-7.5 | 7.0 | 7.0 | 7.0 | 7.0 | 6.9 | 6.9 |
| Chlorhexidine | 0.24-0.36 | 0.31 | 0.31 | 0.30 | 0.32 | 0.30 | 0.31 |
| | mg/ml | | | | | | |
| Sodium fluoride | 0.45-0.55 | 0.50 | 0.50 | 0.51 | 0.51 | 0.51 | 0.52 |
| | mg/ml | | | | | | |

TABLE 16
Stability testing for 40° C. - 75% HR

| Test 40° C. - 75% HR | | Months | | | | | |
|---|---|---|---|---|---|---|---|
| Test | Specification | 0 | 1 | 2 | 3 | 6 | 12 |
| Aspect | Colorless liquid | Compliant | Compliant | Compliant | Compliant | Compliant | Compliant |
| Odor | typical | Compliant | Compliant | Compliant | Compliant | Compliant | Compliant |
| pH | 6.5-7.5 | 7.0 | 7.0 | 7.0 | 7.0 | 6.8 | 6.9 |
| Chlorhexidine | 0.24-0.36 | 0.31 | 0.31 | 0.30 | 0.31 | 0.29 | 0.29 |
| | mg/ml | | | | | | |
| Sodium | 0.45-0.55 | 0.50 | 0.50 | 0.51 | 0.51 | 0.51 | 0.52 |
| fluoride | mg/ml | | | | | | |

As shown in Tables 9-12 and 14-16, the liquid oral care compositions of Example 4 maintains a pH within a range of 6.5 to 7.5 when stored at room temperature (25° C.), low temperatures (4° C.), and elevated temperatures (40° C.). This liquid oral care composition also maintains a near constant amount of chlorohexidine, sodium fluoride, and zinc acetate in solution over the tested time frame.

The liquid oral care compositions of the present disclosure offer a great advantage over chlorhexidine compositions that do not include a pH buffer having at least three hydroxyl groups, such as tromethamine. As chlorhexidine degrades over time and precipitates, the pH as well as the concentration of the active ingredient in the formulation decreases, impacting its effectiveness. The liquid oral care compositions of the present disclosure address this problem and allow for the chlorhexidine to remain in solution and for the composition to maintain a constant pH value.

Whereas particular embodiments of this invention have been described above for purposes of illustration, it will be evident to those skilled in the art that numerous variations of the details of the present invention may be made without departing from the invention as defined in the appended claims.

1. A liquid oral care composition, comprising:
an antihalitosis agent comprising chlorhexidine or a salt thereof; and
a buffering agent comprising a pH buffer having at least three hydroxyl groups.

2. The liquid oral care composition of claim 1, wherein the pH buffer comprises tromethamine.

3. The liquid oral care composition of claim 1, wherein the antihalitosis agent further comprises a metal salt.

4. The liquid oral care composition of claim 3, wherein the metal salt comprises a zinc salt.

5. The liquid oral care composition of claim 4, wherein the zinc salt is selected from the group consisting of: zinc lactate, zinc chloride, zinc citrate, zinc acetate, zinc borate, zinc butyrate, zinc carbonate, zinc formate, zinc gluconate, zinc glycerate, zinc glycolate, zinc phosphate, zinc picolinate, zinc proprionate, zinc salicylate, zinc silicate, zinc stearate, zinc tartrate, zinc undecylenate, zinc phosphate, zinc ricinoleate, zinc nitrate, zinc sulfate, and combinations thereof.

6. The liquid oral care composition of claim 1, further comprising an anticaries agent.

7. The liquid oral care composition of claim 6, wherein the anticaries agent comprises a fluoride salt.

8. The liquid oral care composition of claim 7, wherein the fluoride salt is selected from the group consisting of: stannous fluoride, sodium fluoride, potassium fluoride, potassium monofluorophosphate, sodium monoflourophosphate, ammonium monoflourophosphate, sodium flourosilicate, ammonium flourosilicate, amine fluorides such ammonium fluoride, and combinations thereof.

9. The liquid oral care composition of claim 1, wherein the buffering agent is comprised of at least about 50% by weight tromethamine based on the total weight of the buffering agent.

10. The liquid oral care composition of claim 1, wherein the antihalitosis agent comprises a salt of chlorhexidine, and wherein the salt of chlorohexidine is selected from the group consisting of: chlorhexidine diacetate, chlorhexidine dihydrochloride, chlorhexidine digluconate, and combinations thereof.

11. The liquid oral care composition of claim 1, wherein the antihalitosis agent is present in an amount of about 0.005% to about 1% w/w of the liquid oral care composition.

12. The liquid oral care composition of claim 1, wherein the chlorhexidine or salt thereof is present in an amount of about 0.005% to about 0.1% w/w of the liquid oral care composition.

13. The liquid oral care composition of claim 4, wherein the zinc salt is present in an amount of about 0.1% to about 1% w/w of the liquid oral care composition.

14. The liquid oral care composition of claim 6, wherein the anticaries agent is present in an amount of about 0.01% to about 0.1% w/w of the liquid oral care composition.

15. The liquid oral care composition of claim 1, wherein the buffering agent is present in an amount of about 0.1% to about 5% w/w of the liquid oral care composition.

16. The liquid oral care composition of claim 2, wherein tromethamine or a salt thereof is present in an amount of about 0.1% to about 3% w/w of the liquid oral care composition.

17. The liquid oral care composition of claim 16, wherein tromethamine or a salt thereof is present in an amount of about 0.1% to about 1.21% w/w of the liquid oral care composition.

18. The liquid oral care composition of claim 1, wherein the buffering agent further comprises citric acid.

19. The liquid oral care composition of claim 1, further comprising one or more of a masking agent, a humectant, an emulsifying agent, and a flavoring agent.

20. The liquid oral care composition of claim 19, comprising:
about 0.005% to about 1% w/w of the antihalitosis agent;
about 0.1% to about 5% w/w of the buffering agent;
about 0.01% to about 0.1% w/w of the anticaries agent;
about 0.01% to about 1% of the masking agent;
about 1% to about 25% w/w of the humectant;
about 0.1% to about 10% of the emulsifying agent; and
about 0.1% to about 1% of the flavoring agent.

21. The liquid oral care composition of claim 1, wherein the liquid oral care composition is in the form of a mouthwash, a mouthrinse, or a spray.

22. The liquid oral care composition of claim 1, wherein the liquid oral care composition is an aqueous composition.

23. The liquid oral care composition of claim 1, wherein the liquid oral care composition maintains a pH within a range of about 6.5 to about 7.5 when stored for at least six months at a temperature of about 4° C. to about 40° C.

24. The liquid oral care composition of claim 1, wherein a pH of the liquid oral care composition is maintained within about 0.1 of an original pH value when stored for at least six months at a temperature of about 4° C. to about 40° C.

25. A liquid oral care composition of claim 2, wherein the tromethamine is tromethamine or a salt thereof.

26. (canceled)

27. (canceled)

28. A use of a pH buffer having at least three hydroxyl groups for stabilizing chlorhexidine or a salt thereof in a liquid oral care composition.

29. The use of claim 26 wherein the pH buffer is tromethamine or a salt thereof.
